SEQUENCE LISTING (11 sequences)

SEQ ID NO: 1 - DNA, 25 bp, Artificial
  Synthetic construct
ttttttttttactctaccacatata

SEQ ID NO: 2 - DNA, 22 bp, Artificial
  Synthetic construct
tttttttttttccttgggaacc

SEQ ID NO: 3 - DNA, 23 bp, Artificial
  Synthetic construct
ttttttttttgacagtaaatgcg

SEQ ID NO: 4 - DNA, 22 bp, Artificial
  Synthetic construct
ttttttttttcagcaaatgcca

SEQ ID NO: 5 - DNA, 25 bp, Artificial
  Synthetic construct
ttttttttttaggtcatgaatataa

SEQ ID NO: 6 - DNA, 25 bp, Artificial
  Synthetic construct
tttttttttttacagcaaatatcct

SEQ ID NO: 7 - DNA, 81 bp, Artificial
  Synthetic construct
aggatatttgctgtttatattcatgaccttggcatttgctgcgcatttactgtcggttcccaaggatatatgtggtagagt

SEQ ID NO: 8 - DNA, 86 bp, Artificial
  Synthetic construct
aggatatttgctgtcttatattcatgacctatggcatttgctgacgcatttactgtcaggttcccaaggactatatgtggtagagt

SEQ ID NO: 9 - DNA, 91 bp, Artificial
  Synthetic construct
aggatatttgctgtctttatattcatgacctactggcatttgctgaacgcatttactgtcacggttcccaaggacctatatgtggtagagt

SEQ ID NO: 10 - DNA, 81 bp, Artificial
  Synthetic construct
tggcatttgctgaggatatttgctgtggttcccaaggattatattcatgaccttatatgtggtagagtcgcatttactgtc

SEQ ID NO: 11 - DNA, 10 bp, Artificial
  Synthetic construct
tttttttttt

(19) United States
(12) Patent Application Publication
(10) Pub. No.: US 2024/0209424 A1
(43) Pub. Date: Jun. 27, 2024

(54) Heteromultivalent Spherical Nucleic Acids and Uses in Therapeutic and Diagnostic Applications

(71) Applicant: Emory University, Atlanta, GA (US)

(72) Inventors: Khalid Salaita, Atlanta, GA (US); Brendan Deal, Atlanta, GA (US)

(21) Appl. No.: 17/909,844

(22) PCT Filed: Mar. 9, 2021
(86) PCT No.: PCT/US2021/021465

§ 371 Date: Sep. 7, 2022

Related U.S. Application Data
(60) Provisional application No. 62/987,267, filed on Mar. 9, 2020.

(51) Int. Cl. C12Q 1/6834; A61K 9/14; A61K 45/06; B82Y 5/00

(52) U.S. Cl. CPC C12Q 1/6834; A61K 9/146; A61K 45/06; B82Y 5/00

(57) ABSTRACT

This disclosure relates to spherical nucleic acids comprising a group of nucleic acids that hybridize separate segments of a target nucleic acid for therapeutic and diagnostic applications. In certain embodiments, this disclosure relates to patterning spherical nucleic acids in tandem for hetero-multivalent hybridization to segments of a target nucleic acid.

### CROSS-REFERENCE TO RELATED APPLICATIONS

This application claims the benefit of U.S. Provisional Application No. 62/987,267 filed Mar. 9, 2020. The entirety of this application is hereby incorporated by reference for all purposes.

### INCORPORATION-BY-REFERENCE OF MATERIAL SUBMITTED AS A TEXT FILE VIA THE OFFICE ELECTRONIC FILING SYSTEM (EFS-WEB)

The Sequence Listing associated with this application is provided in text format in lieu of a paper copy and is hereby incorporated by reference into the specification. The name of the text file containing the Sequence Listing is 20064PCT_ST25.txt. The text file is 3 KB, was created on Mar. 8, 2021, and is being submitted electronically via EFS-Web.

### STATEMENT REGARDING FEDERALLY SPONSORED RESEARCH OR DEVELOPMENT

This invention was made with government support under HL142866 awarded by the National Institutes of Health. The government has certain rights in the invention.

### BACKGROUND

Attachment of densely packed spherically oriented oligonucleotides around a nanoparticle core are referred to as spherical nucleic acids (SNAs). SNAs are being investigated and implemented in a variety of applications including diagnostics, anti-sense therapy, and adoptive cell therapy. See e.g., WO 2016/149323, WO 2018/209270, WO 2019/246409, and WO 2019/070890. Many of these applications arise due to desirable properties of SNAs such as improved cellular uptake, nuclease resistance, and binding affinity when compared to linear nucleic acids.

Fong et al. report the role of structural enthalpy in spherical nucleic acid hybridization. J Am Chem Soc, 2018, 140, 6226-6230.

Edwardson et al. report the transfer of molecular recognition information from DNA nanostructures to gold nanoparticles, Nature Chemistry, 2016, 8:162-170.

Estirado et al. report multivalent ultrasensitive interfacing of supramolecular 1D nanoplatforms. J. Am. Chem. Soc. 2019, 141, 18030-18037.

References cited herein are not an admission of prior art.

### SUMMARY

This disclosure relates to spherical nucleic acids comprising a group of nucleic acids that hybridize separate segments of a target nucleic acid for therapeutic and diagnostic applications. In certain embodiments, this disclosure relates to spherical nucleic acids patterned in tandem for hetero-multivalent hybridization to segments of a target nucleic acid. In certain embodiments, this disclosure relates to methods for controlling the relative position of a series of unique oligonucleotides on a nanoparticle surface.

In certain embodiments, this disclosure relates to nanoparticles comprising multiple single stranded block nucleic acids, wherein the multiple single stranded block nucleic acids contain a first segment and a second segment providing multiple first block segments and multiple block second segments, wherein the multiple first block segments contain sequences which are not the same or are substantially variant sequences and hybridize with multiple target segments, and the multiple second block segments are conjugated to the surface of the nanoparticle. In certain embodiments, multiple target segments are contained within a single stranded nucleic acid. In certain embodiments, the multiple single stranded block nucleic acids are in close proximity in a sequential pattern. In certain embodiments, the multiple first block segments are three, four or more than four substantially variant sequences. In certain embodiments, the multiple single stranded block nucleic acids are less than 30, 40 or 50 nucleotides in length. In certain embodiments, the nanoparticle comprises a therapeutic agent. In certain embodiments, the target segments are within a single mRNA or microRNA.

In certain embodiments, this disclosure relates to methods of attaching multiple single stranded block nucleic acids to a nanoparticle surface in close proximity in a sequential pattern comprising: i) providing a nucleic acid complex comprising 1) a single stranded template nucleic acid having multiple template segments and 2) multiple single stranded block nucleic acids, wherein the multiple single stranded block nucleic acids contain a first segment and a second segment providing multiple first block segments and multiple block second segments, wherein the multiple first block segments are substantially variant sequences and hybridize with the multiple template segments, and wherein the multiple second block segments comprise an anchor or functional group for conjugating the multiple single stranded block nucleic acids to the surface of the nanoparticle; ii) mixing the nucleic acid complex with the nanoparticle under conditions such that the second block segments are conjugated to the nanoparticle surface, e.g., functional groups for attaching to the surface of the nanoparticle react with or interact with the nanoparticle surface, providing a nanoparticle coated with the nucleic acid complex; and iii) separating the target sequence from nucleic acid complex providing a nanoparticle coated with multiple single stranded block nucleic acids of substantially variant sequences in close proximity in a sequential pattern.

In certain embodiments, it is contemplated that the block nucleic acids can be covalently or non-covalently anchored, e.g., such that the block nucleic acids have minimal lateral mobility to preserve the spatial pattern. For example, one can non-covalently anchor the block nucleic acids by using cholesterol as an anchor conjugating the block nucleic acids to a nanoparticle surface, wherein the hydrophobic cholesterol inserts into a lipid membrane on the nanoparticle surface wherein the lipid membrane is at non-fluid temperatures, i.e., below the melting transition of the lipid so that there is minimal lateral mobility preserving the spatial pattern. In other contemplated constructions, the anchor can be a specific binding agent such as a ligand and the nanoparticle can be coated with a target molecule, polypeptide, or receptor to a ligand. In certain embodiments the specific binding agent may be another nucleic acid conjugated to the nanoparticle surface that hybridizes with a nucleic acid anchor.

In certain embodiments, separating the target sequence from nucleic acid complex is exposing the nanoparticle coated with the nucleic acid complex with increasing concentrations of buffered salt solutions providing a salt-aged nanoparticle coated with the nucleic acid complex; and exposing the salt-aged nanoparticle coated with the nucleic acid complex with a solution disrupting hybridization providing a nanoparticle coated with multiple single stranded block nucleic acids that are in close proximity in a sequential pattern.

In certain embodiments, the multiple first block segments are three, four, or more than four substantially variant sequences. In certain embodiments, said increasing concentrations of buffered salt solutions is increasing to a maximum concentration of 0.7 M salt solution.

In certain embodiments, separating the target sequence from nucleic acid complex is exposing the nanoparticle coated with the nucleic acid complex to temperatures at or below 0 degrees Celsius providing a frozen nanoparticle coated with the nucleic acid complex; and exposing the frozen nanoparticle coated with the nucleic acid complex to a temperature above 0 degrees Celsius providing a frozen-thawed nanoparticle coated with the nucleic acid complex and exposing the frozen-thawed nanoparticle coated with the nucleic acid complex with a solution disrupting hybridization providing a nanoparticle coated with multiple single stranded block nucleic acids that are in close proximity in a sequential pattern.

In certain embodiments, exposing the nanoparticle coated with the nucleic acid complex to temperatures at or below 0 degrees Celsius provides for the formation of ice in an aqueous solution. In certain embodiments, exposing the nanoparticle coated with the nucleic acid complex to temperatures at or below 0 degrees Celsius is by placing in a −20° C. freezer. In certain embodiments, exposing the nanoparticle coated with the nucleic acid complex to temperatures at or below 0 degrees Celsius is by immersing an aqueous solution into dry ice or liquid nitrogen.

In certain embodiments, this disclosure relates to methods of attaching or printing multiple single stranded block nucleic acids to a nanoparticle surface in close proximity in a sequential pattern comprising: i) providing a nucleic acid complex comprising 1) a single stranded template nucleic acid having multiple template segments and 2) multiple single stranded block nucleic acids, wherein the multiple single stranded block nucleic acids contain a first segment and a second segment providing multiple first block segments and multiple block second segments, wherein the multiple first block segments hybridize with the multiple template segments, and wherein the number of multiple first block segments is three, four or more substantially variant sequences, wherein the multiple second block segments comprise an anchor or functional group for attaching to the surface of the nanoparticle; ii) mixing the nucleic acid complex with the nanoparticle under conditions such that the functional group for attaching to the surface of the nanoparticle reacts with the nanoparticle surface providing a nanoparticle coated with the nucleic acid complex; iii) exposing the nanoparticle coated with the nucleic acid complex with increasing concentrations of buffered salt solutions providing a salt-aged nanoparticle coated with the nucleic acid complex; and iv) exposing the salt-aged nanoparticle coated with the nucleic acid complex with a solution disrupting hybridization providing a nanoparticle coated with multiple single stranded block nucleic acids that are in close proximity in a sequential pattern.

In certain embodiments, this disclosure relates to methods of attaching or printing multiple single stranded block nucleic acids to a nanoparticle surface in close proximity in a sequential pattern comprising: i) providing a nucleic acid complex comprising 1) a single stranded template nucleic acid having multiple template segments and 2) multiple single stranded block nucleic acids, wherein the multiple single stranded block nucleic acids contain a first segment and a second segment providing multiple first block segments and multiple block second segments, wherein the multiple first block segments hybridize with the multiple template segments, and wherein the number of multiple first block segments is three, four or more substantially variant sequences, wherein the multiple second block segments comprise an anchor or a functional group for attaching to the surface of the nanoparticle; ii) mixing the nucleic acid complex with the nanoparticle under conditions such that the anchor conjugates the nucleic acid complex to the surface of the nanoparticle or a functional group for attaching to the surface of the nanoparticle reacts with the nanoparticle surface providing a nanoparticle coated with the nucleic acid complex; iii) exposing the nanoparticle coated with the nucleic acid complex to a temperature at or below 0 degrees Celsius providing a frozen nanoparticle coated with the nucleic acid complex, exposing the nanoparticle coated with the nucleic acid complex to a temperature above 0 degrees Celsius providing a frozen-thawed nanoparticle coated with the nucleic acid complex; and iv) exposing the frozen-thawed nanoparticle coated with the nucleic acid complex with a solution disrupting hybridization providing a nanoparticle coated with multiple single stranded block nucleic acids that are in close proximity in a sequential pattern.

In certain embodiments, the multiple single stranded block nucleic acids are less than 30, 40, or 50 nucleotides in length. In certain embodiments, the multiple first block segments contain three, four, five, or more sequences that are not identical in sequence to each other or are substantially variant sequences.

In certain embodiments, the multiple template segments are separated from each other by one, two, or more nucleobases on the contiguous single stranded template. In certain embodiments, the multiple template segments are separated from each other by less than four nucleobases on the contiguous single stranded template. In certain embodiments, the single stranded template nucleic acid comprises more than 50, 100, 150, 200, 250, or 300 nucleotides. In certain embodiments, the single stranded template nucleic acid comprises less than 100, 150, 200, 250, 300, 400, or 500 nucleotides.

In certain embodiments, the nanoparticle core has a diameter of less than 25, 50, or 100 nm. In certain embodiments, the nanoparticle core has a diameter of more than 5, 10, or 20 nm. In certain embodiments, the nanoparticle is gold and the functional group for attaching to the surface is thiol.

In certain embodiments, this disclosure relates to methods of detecting a single stranded nucleic acid comprising: i) exposing a nanoparticle coated with multiple single stranded block nucleic acids made by the processes disclosed herein to a sample suspected of comprising a single contiguous nucleic acid sequence of greater than 50, 60, 70, 80, 90, or 100 nucleotides having substantially the same sequence as the single stranded template nucleic acid and ii) detecting hybridization of the single contiguous nucleic acid sequence to the nanoparticle coated with multiple single stranded block nucleic acids.

In certain embodiments, the said having substantially the same sequence as the single stranded template nucleic acid is greater than 60%, 70%, 80%, 90%, 95%, 97%, 98% or more identity.

In certain embodiments, this disclosure relates to nanoparticles coated with, comprising or consisting essentially of, multiple single stranded block nucleic acids with substantially variant sequences that are, optionally, in close proximity in a sequential pattern. In certain embodiments, the multiple single stranded block nucleic acids are three, four or more than four which are not the same sequence or are substantially variant sequences. In certain embodiments, the nanoparticle comprises a therapeutic agent or biological agent. In certain embodiments, the nanoparticle comprises a therapeutic agent of biologic agent conjugated to the coating of the particle. In certain embodiments, the agent is conjugated to the terminal end of nucleic acids on the particle. In certain embodiments, the agent is contained in the core of the nanoparticle.

### BRIEF DESCRIPTION OF THE SEVERAL VIEWS OF THE DRAWINGS

FIG. 1A shows a scheme depicting homoMV SNAs, heteroMV SNAs, and spSNAs binding a target.

FIG. 1B illustrates a perspective of target binding n=4 heteroMV SNA with random segment arrangement (circles=binding segments, black line=target) and a perspective of target binding n=6 heteroMV SNA with patterned segment arrangement.

FIG. 1C shows a scheme depicting design of binding interaction between segments 1-6 and target/template sequence containing spacer regions between 0-2 nucleotides and illustrates a nucleic acid complex comprising 1) a single stranded template nucleic acid having multiple template segments and 2) multiple single stranded block nucleic acids, wherein the multiple single stranded block nucleic acids contain a first segment and a second segment providing multiple first block segments and multiple block second segments, wherein the multiple first block segments are substantially variant sequences and hybridize with the multiple template segments, and wherein the multiple second block segments comprise an anchor segment such as functional group to conjugate the complex to a particle surface.

FIG. 1D shows a schematic illustrating the preparation of template/segments complex. Unreduced T10-segments 1-6 were first annealed to the template at 1.2:1 ratio. Following hybridization, thiol protecting groups on segments 1-6 were cleaved using 100× TCEP for ˜30 min. Finally, template/segments complex was purified using a 30k amicon filter to remove unbound segments 1-6, thiol protecting group, and TCEP.

FIG. 1E is a table showing binding sequences for segments 1-6 as well as the target/template with spaces s=0, 1, and 2, SEQ ID NOs: 1-11.

FIGS. 2A-E illustrate the effect of random heteromultivalency on binding valency and binding uniformity.

FIG. 2A shows a schematic illustration describing the melting experiment where excess FAM-labeled target was hybridized to random n=1-6 SNAs and fluorescence was measured as the complex was thermally melted. Inset illustrates how the melting temperature (Tm) and the full width at half-maximum (fwhm) were calculated.

FIG. 2B shows the impact of increasing n on Tm after hybridizing target to SNA in nonstringent buffer (1×PBS).

FIG. 2C shows the fwhm.

FIG. 2D shows data on the impact of increasing n on Tm after hybridizing target to SNA in stringent buffer (0.1×SSC, 0.2% Tween20).

FIG. 2E shows the fwhm.

FIGS. 3A-E illustrate the effect of random heteromultivalency on thermodynamics and affinity.

FIG. 3A shows a schematic illustration describing the experiment to obtain thermodynamic parameters and affinity values. Random n=1-6 SNAs were bound to Cy5-labeled target in 1×SSC, 0.2% Tween20 and the Tm was measured across a range of [SNA+target] (CT) values.

FIG. 3B shows Linear van′t Hoff plots from which thermodynamic values were extracted.

FIG. 3C shows data on ΔH values of random n=1-6 SNAs binding to target.

FIG. 3D shows data on −TΔS values of random n=1-6 SNAs binding to target.

FIG. 3E shows data on −ΔG and log(Keq) values of random n=1-6 SNAs binding to target.

FIGS. 4A-E show data for characterization and binding analysis of patterned heteroMV SNAs.

FIG. 4A is a schematic illustration depicting the synthesis of patterned SNAs. After preannealing segments 1-6 to the template, the complex was incubated with the AuNP. Next, salt aging was performed, and then the template was dehybridized.

FIG. 4B shows quantifying templates bound per SNA before and after dehybridizing.

FIG. 4C shows data on targets bound per AuNP for patterned and mispatterned SNAs after high-stringency washes.

FIG. 4D shows van′t Hoff plots for patterned, random, and mispatterned n=6 SNAs binding the no-spacer target.

FIG. 4E shows −ΔG and log(Keq). Dashed line in represents the predicted −ΔG value (123 kcal/mol) for the non-nicked 81 bp duplex binding in solution.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. In this specification and in the claims that follow, reference will be made to a number of terms that shall be defined to have the following meanings unless a contrary intention is apparent.

As used in this disclosure and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) have the meaning ascribed to them in U.S. Patent law in that they are inclusive or open-ended and do not exclude additional, unrecited elements or method steps.

“Consisting essentially of” or “consists of” or the like, when applied to methods and compositions encompassed by the present disclosure refers to compositions like those disclosed herein that exclude certain prior art elements to provide an inventive feature of a claim, but which may contain additional composition components or method steps, etc., that do not materially affect the basic and novel characteristic(s) of the compositions or methods, compared to those of the corresponding compositions or methods disclosed herein.

As used herein, the terms “substantially variant sequences” refer to a comparison of two nucleotide sequences for which there is less than 10% sequence identity. In certain embodiments, substantially variant sequences will have less than 5% sequence identity. In certain embodiments, substantially variant sequences will be in the context of sequences that have at total length of less than 80, 70, 60, 50, 40, 30, or 20 bases but more than a total length of 6, 7, 8, 10 or 15 bases.

The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity. If relevant, the number of exactly matching bases (expressed as a percentage) in a sequence alignment between two sequences of the alignment calculated using the number of identical positions divided by the greater of the shortest sequence or the number of equivalent positions excluding overhangs wherein internal gaps are counted as an equivalent position.

As used herein, the term “conjugated” refers to linking molecular entities through covalent bonds, i.e. directly or through a linking group, or by other specific binding interactions, such as due to hydrogen bonding and other van der Walls forces. The force to break a covalent bond is high, e.g., about 1500 pN for a carbon to carbon bond. The force to break a combination of strong protein interactions is typically a magnitude less, e.g., biotin to streptavidin is about 150 pN. Thus, a skilled artisan would understand that conjugation must be strong enough to bind molecular entities in order to implement the intended results.

The term “specific binding agent” refers to a molecule, such as a proteinaceous molecule, that binds a target molecule with a greater affinity than other random molecules or proteins. Examples of specific binding agents include an antibody that bind an epitope of an antigen or a receptor which binds a ligand. In certain embodiments, “Specifically binds” refers to the ability of a specific binding agent (such as an ligand, receptor, enzyme, antibody or binding region/fragment thereof) to recognize and bind a target molecule or polypeptide, such that its affinity (as determined by, e.g., affinity ELISA or other assays) is at least 10 times as great, but optionally 50 times as great, 100, 250 or 500 times as great, or even at least 1000 times as great as the affinity of the same for any other or other random molecule or polypeptide.

As used herein, the term “ligand” refers to any organic molecule, i.e., substantially comprised of carbon, hydrogen, and oxygen, that specifically binds to a “receptor.” Receptors are organic molecules typically found on the surface of a cell. Through binding a ligand to a receptor, the cell has a signal of the extra cellular environment which may cause changes inside the cell. As a convention, a ligand is usually used to refer to the smaller of the binding partners from a size standpoint, and a receptor is usually used to refer to a molecule that spatially surrounds the ligand or portion thereof. However as used herein, the terms can be used interchangeably as they generally refer to molecules that are specific binding partners. For example, a glycan may be expressed on a cell surface glycoprotein and a lectin protein may bind the glycan. As the glycan is typically smaller and surrounded by the lectin protein during binding, it may be considered a ligand even though it is a receptor of the lectin binding signal on the cell surface. An antibody may be a receptor, and the epitope may be considered the ligand. In certain embodiments, a ligand is contemplated to be a compound that has a molecular weight of less than 500 or 1,000. In certain embodiments, a receptor is contemplated to be a protein-based compound that has a molecular weight of greater than 1,000, 2,000 or 5,000. In any of the embodiments disclosed herein the position of a ligand and a receptor may be switched.

The terms, “nucleic acid,” or “oligonucleotide,” refer to a polymer of nucleotides. The term “nucleotide” or its plural as used herein is interchangeable with modified forms as discussed herein and otherwise known in the art. In certain instances, the art uses the term “nucleobase” which embraces naturally-occurring nucleotide and non-naturally-occurring nucleotides which include modified nucleotides. Thus, nucleotide or nucleobase means the naturally occurring nucleobases A, G, C, T, and U and non-naturally occurring nucleobases, for example and without limitations, xanthine, diaminopurine, 8-oxo-N6-methyladenine, 7-deazaxanthine, 7-deazaguanine, N′,N′-ethano-2,6-diaminopurine, 5-methylcytosine (mC), 5-(C₃-C₆)-alkynyl-cytosine, 5-fluorouracil, 5-bromouracil, pseudoisocytosine, 2-hydroxy-5-methyl-4-tr-iazolopyridin, isocytosine, isoguanine, and inosine.

Methods of making oligonucleotides of a predetermined sequence are well-known. Solid-phase synthesis methods are preferred for both ribonucleotides and deoxyribonucleotides (the well-known methods of synthesizing DNA are also useful for synthesizing RNA). Ribonucleotides can also be prepared enzymatically.

### Spherical Nucleic Acids (SNAs)

Spherical nucleic acids (SNAs) comprise dense oligonucleotides on the surface of a core particle. The particle can either be organic (e.g., a liposome) inorganic (e.g., gold, silver, or platinum) or hollow (e.g., silica-based). The spherical architecture of the oligonucleotides confers unique advantages over traditional nucleic acid delivery methods, including entry into cells independent of transfection agents and resistance to nuclease degradation. Furthermore, certain SNAs can penetrate biological barriers, including the blood-brain and blood-tumor barriers as well as the epidermis.

In certain embodiments, this disclosure contemplates SNAs particles or nanoparticles having multiple heteromultivalent oligonucleotides which are optionally attached to nanoparticles in a pattern (patterned SNAs) by using methods disclosed herein. In general, the core nanoparticle of SNAs or patterned SNAs are contemplated to include any compound or substance with a high loading capacity for oligonucleotides as described herein, including for example and without limitation, a metal, a semiconductor, a liposomal particle, insulator particle compositions, and a dendrimer (organic versus inorganic). It is also contemplated that the core structure could be removed, e.g., by metal or salt leaching, to leave the patterned SNAs in relative proximity to each other.

In practice, methods of increasing cellular uptake and inhibiting gene expression are provided using any suitable particle having oligonucleotides attached thereto that do not interfere with complex formation, i.e., hybridization to a target nucleic acid. The size, shape and chemical composition of the particles contribute to the properties of the resulting oligonucleotide-functionalized nanoparticle. These properties include for example, optical properties, optoelectronic properties, electrochemical properties, electronic properties, stability in various solutions, magnetic properties, and pore and channel size variation. The use of mixtures of particles having different sizes, shapes and/or chemical compositions, as well as the use of nanoparticles having uniform sizes, shapes, and chemical composition, is contemplated. Examples of suitable particles include, without limitation, nanoparticles, aggregate particles, isotropic (such as spherical particles) and anisotropic particles (such as non-spherical rods, tetrahedral, prisms) and core-shell particles. Methods of making metal, semiconductor and magnetic nanoparticles are well-known in the art.

Thus, SNAs or patterned SNAs are contemplated which comprise a variety of inorganic materials including, but not limited to, metals, semi-conductor materials, organic materials, or ceramics. In certain embodiments, the particle is metallic, and in various embodiments, the particle is a colloidal metal. Thus, in various embodiments, particles useful in the practice of the methods include metal (including for example and without limitation, gold, silver, platinum, aluminum, palladium, copper, cobalt, indium, nickel, or any other metal amenable to nanoparticle formation), semiconductor (including for example and without limitation, CdSe, CdS, and CdS or CdSe coated with ZnS) and magnetic colloidal materials. In certain embodiments, metal-based particles include those described herein.

Ceramic nanoparticle materials include, but are not limited to, brushite, tricalcium phosphate, alumina, silica, and zirconia. Organic materials from which nanoparticles are produced include carbon-based nanoparticle polymers such as polystyrene, silicone rubber, polycarbonate, polyurethanes, polypropylenes, polymethylmethacrylate, polyvinyl chloride, polyesters, polyethers, and polyethylene. Biodegradable, biopolymer (e.g., polypeptides such as BSA, polysaccharides, etc.), other biological materials (e.g., carbohydrates), and/or polymeric compounds are also contemplated for use in producing particles.

Liposomal particles are also contemplated by the disclosure. Hollow particles are also contemplated herein. Liposomal particles of the disclosure have at least a substantially spherical geometry, an internal side and an external side, and comprise a lipid bilayer. The lipid bilayer comprises, in various embodiments, a lipid from the phosphocholine family of lipids or the phosphoethanolamine family of lipids. While not meant to be limiting, a lipid or combination of lipids is chosen from group consisting of 1,2-dioleoyl-sn-glycero-3-phosphocholine (DOPC), 1,2-dimyristoyl-sn-phosphatidylcholine (DMPC), 1-palmitoyl-2-oleoyl-sn-phosphatidylcholine (POPC), 1,2-distearoyl-sn-glycero-3-phospho-(l′-rac-glycerol) (DSPG), 1,2-dioleoyl-sn-glycero-3-phospho-(l′-rac-glycerol) (DOPG), 1,2-distearoyl-sn-glycero-3-phosphocholine (DSPC), 1,2-dipalmitoyl-sn-glycero-3-phosphocholine (DPPC), 1,2-di-(9Z-octadecenoyl)-sn-glycero-3-phosphoethanolamine (DOPE), 1,2-dihexadecanoyl-sn-glycero-3-phosphoethanolamine (DPPE), cardiolipin, lipid A, and a combination thereof.

SNAs or patterned SNAs contemplated by the disclosure can range in size from about 1 nm to about 250 nm in mean diameter, about 1 nm to about 240 nm in mean diameter, about 1 nm to about 230 nm in mean diameter, about 1 nm to about 220 nm in mean diameter, about 1 nm to about 210 nm in mean diameter, about 1 nm to about 200 nm in mean diameter, about 1 nm to about 190 nm in mean diameter, about 1 nm to about 180 nm in mean diameter, about 1 nm to about 170 nm in mean diameter, about 1 nm to about 160 nm in mean diameter, about 1 nm to about 150 nm in mean diameter, about 1 nm to about 140 nm in mean diameter, about 1 nm to about 130 nm in mean diameter, about 1 nm to about 120 nm in mean diameter, about 1 nm to about 110 nm in mean diameter, about 1 nm to about 100 nm in mean diameter, about 1 nm to about 90 nm in mean diameter, about 1 nm to about 80 nm in mean diameter, about 1 nm to about 70 nm in mean diameter, about 1 nm to about 60 nm in mean diameter, about 1 nm to about 50 nm in mean diameter, about 1 nm to about 40 nm in mean diameter, about 1 nm to about 30 nm in mean diameter, or about 1 nm to about 20 nm in mean diameter, about 1 nm to about 10 nm in mean diameter. In other aspects, the size of the nanoparticles is from about 5 nm to about 150 nm (mean diameter), from about 5 to about 50 nm, from about 10 to about 30 nm, from about 10 to 150 nm, from about 10 to about 100 nm, or about 10 to about 50 nm. The size of the nanoparticles is from about 5 nm to about 150 nm (mean diameter), from about 30 to about 100 nm, from about 40 to about 80 nm. The size of the nanoparticles used in a method varies as required by their particular use or application. The variation of size is advantageously used to optimize certain physical characteristics of the nanoparticles, for example, optical properties or the amount of surface area that can be functionalized as described herein. In further embodiments, a plurality of SNAs or patterned SNAs is produced and the SNAs in the plurality have a mean diameter of less than or equal to about 50 nanometers (e.g., about 5 nanometers to about 50 nanometers, or about 5 nanometers to about 40 nanometers, or about 5 nanometers to about 30 nanometers, or about 5 nanometers to about 20 nanometers, or about 10 nanometers to about 50 nanometers, or about 10 nanometers to about 40 nanometers, or about 10 nanometers to about 30 nanometers, or about 10 nanometers to about 20 nanometers). In further embodiments, the SNAs in the plurality created by a method of the disclosure have a mean diameter of less than or equal to about 20 nanometers, or less than or equal to about 25 nanometers, or less than or equal to about 30 nanometers, or less than or equal to about 35 nanometers, or less than or equal to about 40 nanometers, or less than or equal to about 45 nanometers.

In certain embodiments, this disclosure contemplates SNAs or patterned SNAs that are functionalized with oligonucleotides or a modified form thereof generally comprise an oligonucleotide from about 5 nucleotides to about 100 nucleotides in length. Accordingly, oligonucleotides of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, about 125, about 150, about 175, about 200, about 250, about 300, about 350, about 400, about 450, about 500 or more nucleotides in length are contemplated.

In some embodiments, the oligonucleotides attached to a SNAs or patterned SNAs are DNA. When DNA is attached to the nanoparticle, the DNA is, in some embodiments, comprised of sequences of sufficiently complementary to target regions of oligonucleotides such that hybridization of the oligonucleotides attached to a nanoparticle and the target polynucleotide take place, thereby associating the target polynucleotide to the nanoparticle. The oligonucleotides in various aspects are single stranded. In some embodiments, single stands may be made double-stranded, as long as the double-stranded molecule also includes a single strand region that hybridizes to a single strand region of the target polynucleotide.

In some embodiments, the disclosure contemplates that oligonucleotides attached to SNAs or patterned SNAs are RNA. The RNA can be either single-stranded or double-stranded, so long as it is able to hybridize to a target polynucleotide.

In certain embodiments, all of the multiple oligonucleotides have different sequences or variant sequences providing for hybridization to variant target sequences (heteromultivalent). In certain embodiments, variant sequences have less than 10%, 5%, 2% or 1% identity to each other. In further aspects, multiple oligonucleotides are arranged in tandem and are separated by a spacer.

Oligonucleotides contemplated for use in the methods disclosed herein include those bound to the nanoparticle through any means (e.g., covalent or non-covalent attachment). Attachment in various aspects is effected through a 5′ linkage, a 3′ linkage, some type of internal linkage, or any combination of these attachments. In some embodiments, the oligonucleotides are covalently attached to a nanoparticle. In further embodiments, the oligonucleotides are non-covalently attached to a nanoparticle.

In certain embodiment, this disclosure contemplates the use of molecules that prevent opsonization. For example, and without limitation, the disclosure contemplates the use of polysaccharides (e.g., dextran) or polyethylene glycol (PEG).

An oligonucleotide surface density adequate to make the nanoparticles stable and the conditions necessary to obtain it for a desired combination of nanoparticles and polynucleotides can be determined empirically. The density of oligonucleotides on the surface of the SNA or patterned SNA is measured by the number of oligonucleotides on the surface. With respect to the surface density of oligonucleotides on the surface of a SNA or patterned SNA of the disclosure, it is contemplated that a SNA or patterned SNA as described herein comprises from about 1 to about 300 oligonucleotides on its surface. In various embodiments, a SNA or patterned SNA comprises from about 10 to about 300, or from about 10 to about 250, or from about 10 to about 200, or from about 10 to about 150, or from about 10 to about 100, or from 10 to about 90, or from about 10 to about 80, or from about 10 to about 70, or from about 10 to about 60, or from about 10 to about 50, or from about 10 to about 40, or from about 10 to about 30, or from about 10 to about 20 oligonucleotides on its surface.

In certain aspects, SNAs or patterned SNAs are contemplated which include those wherein oligonucleotides are 5′ or 3′ terminally conjugated to a spacer moiety, e.g., polyethylene glycol (PEG), a saccharide or polysaccharide, or a peptide-PEG moiety. A water solubilizing moiety may be attached to the nanoparticle through a “spacer.” “Spacer” as used herein is a moiety that serves to increase distance between the nanoparticle and oligonucleotides or between oligonucleotides and outer moieties. In further aspects, the spacer is a polymer, including but not limited to a water-soluble polymer, a nucleic acid, a polypeptide, an oligosaccharide, a carbohydrate, a lipid, an ethylene glycol, or combinations thereof. In some embodiments, the spacer is PEG.

In certain aspects, the oligonucleotide has a spacer through which it is covalently bound to the nanoparticles. As a result of the binding of the spacer to the nanoparticles, the oligonucleotides are spaced away from the surface of the nanoparticles. In various embodiments, the length of the spacer is or is equivalent to at least about 5 nucleotides, 5-10 nucleotides, 10 nucleotides, 10-30 nucleotides, or even greater than 30 nucleotides. The spacer may have any sequence which does not interfere with the ability of the polynucleotides to become bound to the nanoparticles or to a target nucleic acid. In certain aspects, the bases of a nucleotide spacer are all adenylic acids, all thymidylic acids, all cytidylic acids, all guanylic acids, all uridylic acids, or all some other modified base.

In certain embodiments, this disclosure contemplates using a cleavable linker to attach PEG to the oligonucleotides. In some embodiments, the cleavable linker comprises a peptide sequence that is recognized and cleaved by a specific enzyme. The use of a cleavable peptide linker sequence allows for the generation of SNAs that possess the properties of increased in vivo circulation time while maintaining high cellular uptake. In addition, the programmability of PEG cleavage (e.g., via modulating peptide sequence, PEG density, and/or PEG length) can be used to effectuate desired therapeutic applications.

It is contemplated that in some embodiments, SNAs or patterned SNAs of the disclosure possess the ability to regulate gene expression. The oligonucleotides utilized in the synthesis of the SNAs or patterned SNAs include heteromultivalent specific sequences that can be used to regulate the expression of specific proteins by cells in order to modulate cell behavior (e.g., slow proliferation, induce cell death). Thus, in some embodiments, a SNA or patterned SNA of the disclosure comprises oligonucleotides having gene regulatory activity (e.g., inhibition of target gene expression or target cell recognition).

In various aspects, this disclosure contemplates methods using heteromultivalent oligonucleotide segments that are optionally when arranged in tandem are 100% complementary to a target polynucleotide, i.e., a perfect match, while in other aspects, heteromultivalent oligonucleotide segments that when arranged in tandem are about or at least (meaning greater than or equal to) about 95% complementary to a target polynucleotide over the length of the target polynucleotide, about or at least about 90%, about or at least about 85%, about or at least about 80%, about or at least about 75%, about or at least about 70%, about or at least about 65%, about or at least about 60%, about or at least about 55%, about or at least about 50%, about or at least about 45%, about or at least about 40%, about or at least about 35%, about or at least about 30%, about or at least about 25%, about or at least about 20% complementary to the target polynucleotide over the length of the target polynucleotide to the extent that the heteromultivalent oligonucleotide segments able to achieve the desired degree of inhibition of a target gene product. The remaining non-complementary nucleotides may be clustered or interspersed with complementary nucleobases and need not be contiguous to each other or to complementary nucleotides. Percent complementarity of an inhibitory oligonucleotide with a region of a target nucleic acid can be determined routinely using BLAST programs (basic local alignment search tools) and PowerBLAST programs known in the art.

Accordingly, methods of utilizing a SNA or patterned SNA of the disclosure in gene regulation therapy are provided. This method comprises the step of hybridizing heteromultivalent oligonucleotide segments to a nucleic acid encoding the gene with all or a portion of the target sequence, wherein hybridizing between the target and the oligonucleotide segments occurs over a length of the target with a degree of complementarity sufficient to inhibit expression of the gene product. The inhibition of gene expression may occur in vivo or in vitro.

In some embodiments, the disclosure contemplates a SNA or patterned SNA or oligonucleotide-functionalized nanoparticle further comprising an agent. In various embodiments, the agent is a peptide, a protein, an antibody, a small molecule, or a combination thereof. In any of the embodiments of the disclosure, the agent is encapsulated in the nanoparticle.

An “agent” as used herein means any compound useful for therapeutic or diagnostic purposes. The term as used herein is understood to include any compound that is administered to a patient for the treatment or diagnosis of a condition.

Protein therapeutic agents include, without limitation peptides, enzymes, structural proteins, receptors and other cellular or circulating proteins as well as fragments and derivatives thereof, the aberrant expression of which gives rise to one or more disorders. Therapeutic agents also include, as one specific embodiment, chemotherapeutic agents. Therapeutic agents also include, in various embodiments, a radioactive material.

In various aspects, protein therapeutic agents include cytokines or hematopoietic factors including without limitation IL-1 alpha, IL-1 beta, IL-2, IL-3, IL-4, IL-5, IL-6, IL-11, colony stimulating factor-1 (CSF-1), M-CSF, SCF, GM-CSF, granulocyte colony stimulating factor (G-CSF), interferon-alpha (IFN-alpha), consensus interferon, IFN-beta, IFN-gamma, IL-7, IL-8, IL-9, IL-10, IL-12, IL-13, IL-14, IL-15, IL-16, IL-17, IL-18, erythropoietin (EPO), thrombopoietin (TPO), angiopoietins, for example Ang-1, Ang-2, Ang-4, Ang-Y, the human angiopoietin-like polypeptide, vascular endothelial growth factor (VEGF), angiogenin, bone morphogenic protein-1, bone morphogenic protein-2, bone morphogenic protein-3, bone morphogenic protein-4, bone morphogenic protein-5, bone morphogenic protein-6, bone morphogenic protein-7, bone morphogenic protein-8, bone morphogenic protein-9, bone morphogenic protein-10, bone morphogenic protein-11, bone morphogenic protein-12, bone morphogenic protein-13, bone morphogenic protein-14, bone morphogenic protein-15, bone morphogenic protein receptor IA, bone morphogenic protein receptor IB, brain derived neurotrophic factor, ciliary neurotrophic factor, ciliary neurotrophic factor receptor, cytokine-induced neutrophil chemotactic factor 1, cytokine-induced neutrophil, chemotactic factor 2a, cytokine-induced neutrophil chemotactic factor 2β, β endothelial cell growth factor, endothelin 1, epidermal growth factor, epithelial-derived neutrophil attractant, fibroblast growth factor 4, fibroblast growth factor 5, fibroblast growth factor 6, fibroblast growth factor 7, fibroblast growth factor 8, fibroblast growth factor 8b, fibroblast growth factor 8c, fibroblast growth factor 9, fibroblast growth factor 10, fibroblast growth factor acidic, fibroblast growth factor basic, glial cell line-derived neurotrophic factor receptor α1, glial cell line-derived neurotrophic factor receptor a2, growth related protein, growth related protein a, growth related protein ß, growth related protein γ, heparin binding epidermal growth factor, hepatocyte growth factor, hepatocyte growth factor receptor, insulin-like growth factor I, insulin-like growth factor receptor, insulin-like growth factor II, insulin-like growth factor binding protein, keratinocyte growth factor, leukemia inhibitory factor, leukemia inhibitory factor receptor a, nerve growth factor nerve growth factor receptor, neurotrophin-3, neurotrophin-4, placenta growth factor, placenta growth factor 2, platelet-derived endothelial cell growth factor, platelet derived growth factor, platelet derived growth factor A chain, platelet derived growth factor AA, platelet derived growth factor AB, platelet derived growth factor B chain, platelet derived growth factor BB, platelet derived growth factor receptor a, platelet derived growth factor receptor β, pre-β cell growth stimulating factor, stem cell factor receptor, TNF, including TNF0, TNF1, TNF2, transforming growth factor a, transforming growth factor β, transforming growth factor β1, transforming growth factor β2, transforming growth factor ß3, transforming growth factor β5, latent transforming growth factor ß1, transforming growth factor ß binding protein I, transforming growth factor ß binding protein II, transforming growth factor ß binding protein III, tumor necrosis factor receptor type I, tumor necrosis factor receptor type II, urokinase-type plasminogen activator receptor, vascular endothelial growth factor, and chimeric proteins and biologically or immunologically active fragments thereof.

Examples of biologic agents include, but are not limited to, immuno-modulating proteins such as cytokines, monoclonal antibodies against tumor antigens, tumor suppressor genes, and cancer vaccines. Examples of interleukins that may be used in conjunction with the compositions and methods of the present invention include, but are not limited to, interleukin 2 (IL-2), and interleukin 4 (IL-4), interleukin 12 (IL-12). Other immuno-modulating agents other than cytokines include, but are not limited to bacillus Calmette-Guerin, levamisole, and octreotide.

In various embodiments, therapeutic agents are contemplated for use in the compositions and methods disclosed herein and include, but are not limited to, alkylating agents, antibiotic agents, antimetabolic agents, hormonal agents, plant-derived agents, and biologic agents.

Examples of alkylating agents include, but are not limited to, bischloroethylamines (nitrogen mustards, e.g. chlorambucil, cyclophosphamide, ifosfamide, mechlorethamine, melphalan, uracil mustard), aziridines (e.g. thiotepa), alkyl sulfonates (e.g. busulfan), nitrosoureas (e.g. carmustine, lomustine, streptozocin), nonclassic alkylating agents (altretamine, dacarbazine, and procarbazine), platinum compounds (e.g., carboplatin, cisplatin and platinum (IV) (Pt(IV))).

Examples of antibiotic agents include, but are not limited to, anthracyclines (e.g. doxorubicin, daunorubicin, epirubicin, idarubicin and anthracenedione), mitomycin C, bleomycin, dactinomycin, plicamycin. Additional antibiotic agents are discussed in detail below.

Examples of antimetabolic agents include, but are not limited to, fluorouracil (5-FU), floxuridine (5-FUdR), methotrexate, leucovorin, hydroxyurea, thioguanine (6-TG), mercaptopurine (6-MP), cytarabine, pentostatin, fludarabine phosphate, cladribine (2-CDA), asparaginase, imatinib mesylate (or GLEEVEC®), and gemcitabine.

Examples of hormonal agents include, but are not limited to, synthetic estrogens (e.g., diethylstibestrol), antiestrogens (e.g. tamoxifen, toremifene, fluoxymesterol and raloxifene), antiandrogens (bicalutamide, nilutamide, flutamide), aromatase inhibitors (e.g., aminoglutethimide, anastrozole and tetrazole), ketoconazole, goserelin acetate, leuprolide, megestrol acetate and mifepristone.

Examples of plant-derived agents include, but are not limited to, vinca alkaloids (e.g., vincristine, vinblastine, vindesine, vinzolidine and vinorelbine), podophyllotoxins (e.g., etoposide (VP-16) and teniposide (VM-26)), camptothecin compounds (e.g., 20(S) camptothecin, topotecan, rubitecan, and irinotecan), taxanes (e.g., paclitaxel and docetaxel).

Chemotherapeutic agents contemplated for use include, without limitation, alkylating agents including: nitrogen mustards, such as mechlorethamine, cyclophosphamide, ifosfamide, melphalan and chlorambucil; nitrosoureas, such as carmustine (BCNU), lomustine (CCNU), and semustine (methyl-CCNU); ethylenimines/methylmelamine such as thriethylenemelamine (TEM), triethylene, thiophosphoramide (thiotepa), hexamethylmelamine (HMM, altretamine); alkyl sulfonates such as busulfan; triazines such as dacarbazine (DTIC); antimetabolites including folic acid analogs such as methotrexate and trimetrexate, pyrimidine analogs such as 5-fluorouracil, fluorodeoxyuridine, gemcitabine, cytosine arabinoside (AraC, cytarabine), 5-azacytidine, 2,2′-difluorodeoxycytidine, purine analogs such as 6-mercaptopurine, 6-thioguanine, azathioprine, 2′-deoxycoformycin (pentostatin), erythrohydroxynonyladenine (EHNA), fludarabine phosphate, and 2-chlorodeoxyadenosine (cladribine, 2-CdA); natural products including antimitotic drugs such as paclitaxel, vinca alkaloids including vinblastine (VLB), vincristine, and vinorelbine, taxotere, estramustine, and estramustine phosphate; epipodophylotoxins such as etoposide and teniposide; antibiotics such as actimomycin D, daunomycin (rubidomycin), doxorubicin, mitoxantrone, idarubicin, bleomycins, plicamycin (mithramycin), mitomycin C, and actinomycin; enzymes such as L-asparaginase; biological response modifiers such as interferon-alpha, IL-2, G-CSF and GM-CSF; miscellaneous agents including platinum coordination complexes such as cisplatin, Pt(IV) and carboplatin, anthracenediones such as mitoxantrone, substituted urea such as hydroxyurea, methylhydrazine derivatives including N-methylhydrazine (MIH) and procarbazine, adrenocortical suppressants such as mitotane and aminoglutethimide; hormones and antagonists including adrenocorticosteroid antagonists such as prednisone and equivalents, dexamethasone and aminoglutethimide; progestin such as hydroxyprogesterone caproate, medroxyprogesterone acetate and megestrol acetate; estrogen such as diethylstilbestrol and ethinyl estradiol equivalents; antiestrogen such as tamoxifen; androgens including testosterone propionate and fluoxymesterone/equivalents; antiandrogens such as flutamide, gonadotropin-releasing hormone analogs and leuprolide; and non-steroidal antiandrogens such as flutamide.

Chemotherapeutics also include, but are not limited to, an anti-PD-1 antibody, alkylating agents, angiogenesis inhibitors, antibodies, antimetabolites, antimitotics, antiproliferatives, antivirals, aurora kinase inhibitors, apoptosis promoters (for example, Bcl-2 family inhibitors), activators of death receptor pathway, Bcr-Abl kinase inhibitors, BiTE (Bi-Specific T cell Engager) antibodies, antibody drug conjugates, biologic response modifiers, Bruton's tyrosine kinase (BTK) inhibitors, cyclin-dependent kinase inhibitors, cell cycle inhibitors, cyclooxygenase-2 inhibitors, DVDs, leukemia viral oncogene homolog (ErbB2) receptor inhibitors, growth factor inhibitors, heat shock protein (HSP)-90 inhibitors, histone deacetylase (HDAC) inhibitors, hormonal therapies, immunological agents, inhibitors of inhibitors of apoptosis proteins (IAPs), intercalating antibiotics, kinase inhibitors, kinesin inhibitors, Jak2 inhibitors, mammalian target of rapamycin inhibitors, microRNAs, mitogen-activated extracellular signal-regulated kinase inhibitors, multivalent binding proteins, non-steroidal anti-inflammatory drugs (NSAIDs), poly ADP (adenosine diphosphate)-ribose polymerase (PARP) inhibitors, platinum chemotherapeutics {e.g., cisplatin), polo-like kinase inhibitors, phosphoinositide-3 kinase (PI3K) inhibitors, proteasome inhibitors, purine analogs, pyrimidine analogs, receptor tyrosine kinase inhibitors, retinoids/deltoids plant alkaloids, topoisomerase inhibitors, ubiquitin ligase inhibitors, and the like, as well as combinations of one or more of these agents.

The disclosure includes compositions that comprise a pharmaceutically acceptable carrier and SNAs or patterned SNAs of the disclosure. In some embodiments, the composition is an antigenic composition. The term “carrier” refers to a vehicle within which the SNAs or patterned SNAs are administered to a mammalian subject. The term carrier encompasses diluents, excipients, adjuvants and combinations thereof.

Exemplary “diluents” include sterile liquids such as sterile water, saline solutions, and buffers (e.g., phosphate, tris, borate, succinate, or histidine). Exemplary “excipients” are inert substances include but are not limited to polymers (e.g., polyethylene glycol), carbohydrates (e.g., starch, glucose, lactose, sucrose, or cellulose), and alcohols (e.g., glycerol, sorbitol, or xylitol).

### Spatial Control of Multivalent Binding in DNA-Nanoparticle Conjugates Leads to Enhanced Affinity

Increasing the length of oligonucleotides on the SNA over around 50 bases has certain disadvantages. For example, increasing the length increases access to nucleases. It also reduces the affinity advantage offered by SNAs because this leads to a reduction in the DNA density and places binding further away from the nanoparticle core. Therefore, simply increasing the oligo length on SNAs will not necessarily improve target binding in terms of affinity. Thus, finding alternate approaches to boosting the affinity of SNAs to long nucleic acid targets is needed.

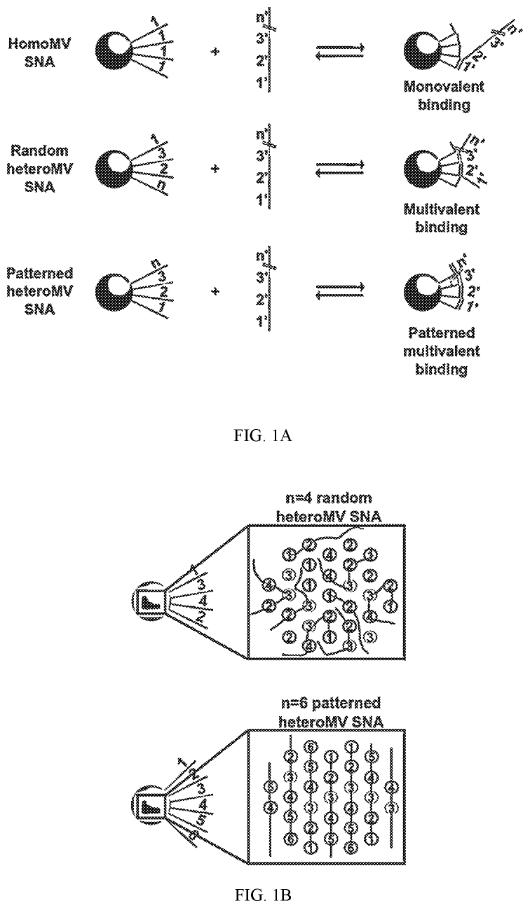

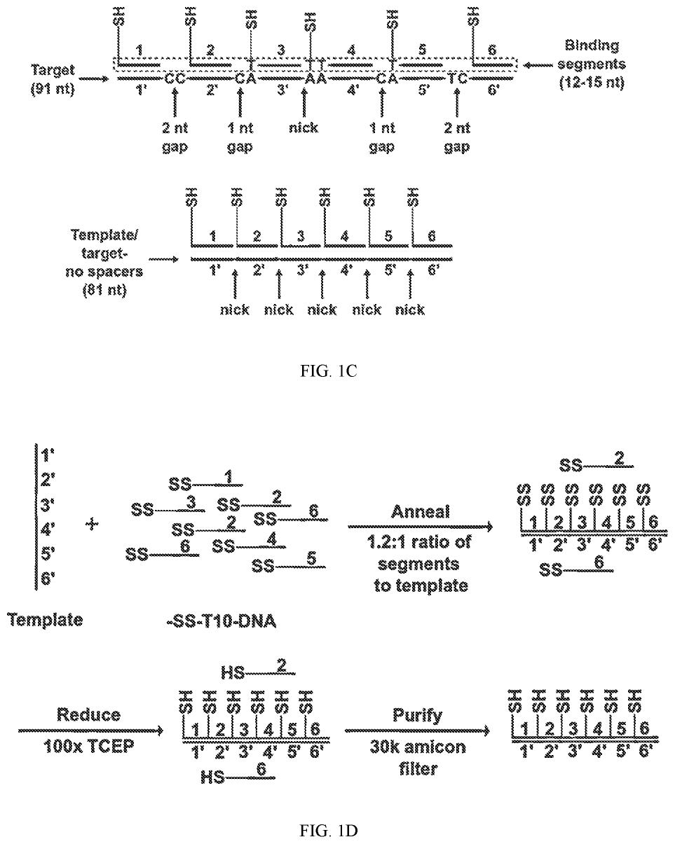

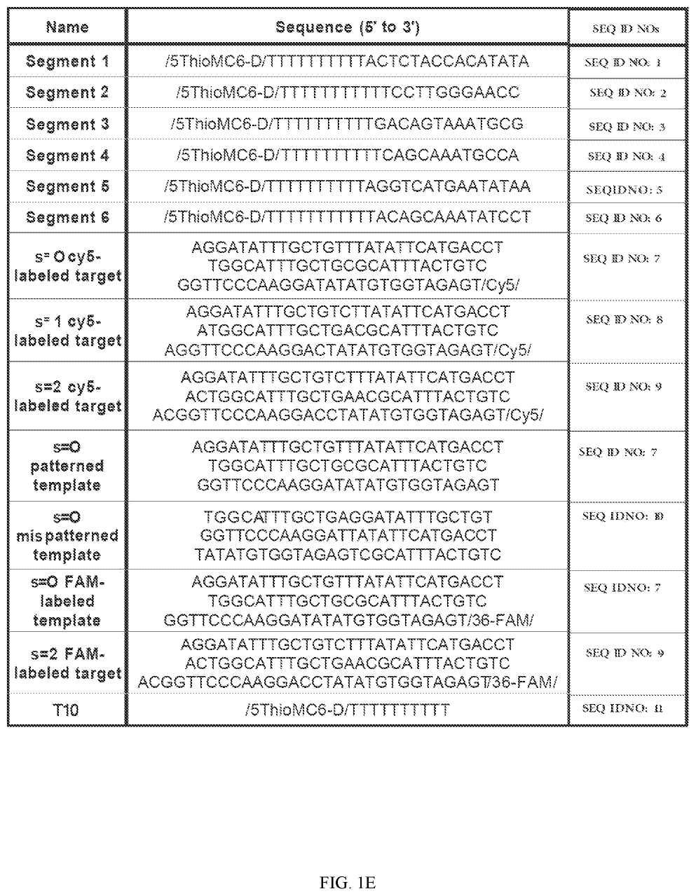

Within multivalent binding, there are two main varieties of interactions, namely homomultivalent (homoMV) and heteromultivalent (heteroMV). HomoMV interactions employ multiple copies of the same ligand and the same receptor. Thus, conventional SNAs are formally homoMV ligands, consisting of many copies of the same oligonucleotide that bind monovalently to a single nucleic acid target. HeteroMV differs from homoMV by employing two or more orthogonal pairs of ligands and receptors that interact concurrently (FIG. 1A).

Experiments were performed to determine whether heteroMV SNAs are advantageous because they offer an approach to bind long nucleic acid targets with high affinity. Testing this hypothesis is especially relevant for hybridizing mRNA and for gene regulation where the SNA is engineered to bind nucleic acids. Another advantage of heteroMV SNAs is that the nanoparticle-DNA conjugate maintains the DNA length and density characteristic of traditional SNAs while still offering improved binding affinity. Recognizing the importance of ligand positioning in multivalency, a second hypothesis that spatial patterning of the DNA oligos on the particle surface can lead to a further enhancement in target affinity was tested. A strategy to create spatially patterned SNAs was developed where the target nucleic acid guides the molecular deposition of complementary oligos on the nanoparticle surface (FIG. 1A).

Reported herein are experiments using heteroMV SNAs comprised of six unique oligo sequences designed to bind an about 90 nt DNA target. By measuring melting properties, data indicates the melting temperature of randomly anchored heteroMV SNAs increases with the inclusion of each additional oligo until a certain point, which is dependent on hybridization conditions, where the random arrangement limits the maximum binding valency. Moreover, data indicates the thermodynamic parameters governing the multivalent interaction. Random heteromultivalency led to an about 50 order of magnitude increase in binding affinity compared to the corresponding homoMV SNA, as a result of decreasing the enthalpic cost of binding. Furthermore, spatial patterning of heteroMV SNAs results in a further about 15 order of magnitude enhancement in the affinity of binding. Overall, the incorporation of heteromultivalency and spatial patterning into the SNA platform offers an approach for fine-tuning SNA binding affinity which improves the diagnostic and therapeutic potential of SNAs.

HeteroMV SNAs were created in order to bind an oligo target multivalently. Specifically, by including multiple unique oligo sequences, each complementary to a specific region of a ssDNA target, multiple oligo segments bind the target simultaneously. Aided by this multivalent binding, a dramatic enhancement in melting temperature and binding affinity was observed relative to homoMV SNAs. This multivalent interaction where multiple oligonucleotides bind a receptor simultaneously is a tertiary level of multivalency in DNA binding. Alternatively, an interaction between two homoMV DNA-particle conjugates or a homoMV DNA-particle and a planar surface coated in complementary DNA does demonstrate tertiary multivalency. The tertiary level of multivalency in DNA hybridization has also been explored between two 1D platforms, revealing a linear increase in binding affinity as the ligand valency increases. However, the tertiary multivalency described herein is distinct in that the receptor is a long (˜90 nt) single-stranded oligo target. Such a target, resembling mRNA or genomic DNA, is important to many applications, including diagnostics and therapeutics. Thus, the enhanced binding avidity to an oligo target presented herein has the potential to heighten the efficacy of SNAs in these important applications. A trade-off between maximizing the binding capacity of a nanoparticle and its binding affinity (quantity versus quality of binding) was identified. For example, under stringent hybridization conditions, fewer targets bind a particle, but these few targets display a greater binding valency. In other words, under stringent conditions, the target can sample a wider range of binding geometries to overcome kinetic barriers and maximize the number of segments bound, thus reaching a thermodynamic-minimum.

Importantly, a 15-order of magnitude enhancement in binding affinity is demonstrate when six complementary oligonucleotides are spatially organized on the particle surface instead of randomly positioned. This massive enhancement is the result of a templating-based strategy on the surface of the nanoparticle. An improvement in binding affinity was observed for the patterned SNAs as flexible spacers were removed between elements. This suggests that the molecular printing strategy is correctly positioning DNA segments and the spacer only diminishes affinity.

Heteromultivalent (heteroMV) Spherical Nucleic Acids (SNA), Have Increased Tm and are Aided by Stringent Hybridization Conditions

Six oligonucleotides (segments 1-6) between 12-15 nucleotides in length with similar melting temperatures (Tm's within about 6° C.) were designed to bind sequentially along a 91 nucleotide DNA target (FIG. 1C, 1D). In order to increase flexibility and reach for the target, a 2 nucleotide gap was included in the target sequence between each segments binding location. There is a complex relationship between flexibility and improved multivalent binding. Too little flexibility or too much flexibility can dampen affinity. Additional flexibility is gained by including gaps between binding regions potentially improving the multivalent binding interactions. Additionally, each of the six 12-15mers (segments 1-6) contain a T₁₀ linker terminated with a thiol group at the 5′ end to allow conjugation to an about 13 nm AuNP core. A series of SNAs were then synthesized with increasing number of distinct sequences (n) using the salt-aging method, beginning with a traditional homoMV SNA containing only segment 1 (n=1 SNA) and ending with a heteroMV SNA incubated with equimolar concentrations of segments 1-6 (n=6 SNA). The total number of oligos per AuNP for n=1 SNA and n=6 SNAs was measured to be about 165 segments/particle for both particle compositions.

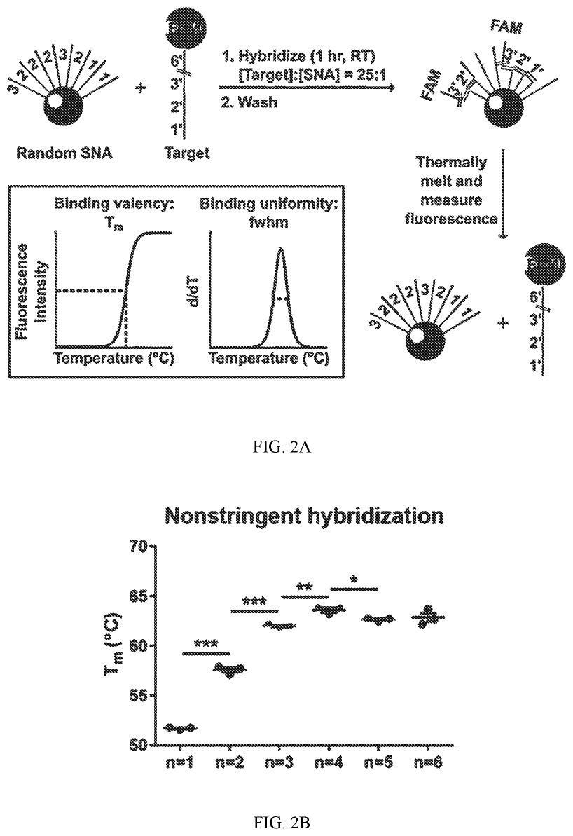

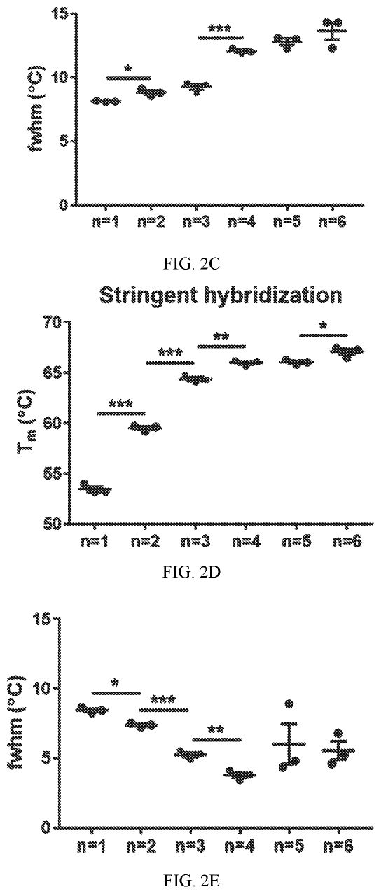

In order to determine if heteroMV SNAs form efficient multivalent interactions with the target sequence, melting curves were measured for the SNA-target complex. First, n=1-6 SNAs were incubated for one hour with a 25-fold excess of a FAM-labeled target sequence in 1× PBS (FIG. 2A). Following hybridization, unbound targets were removed through washing, and the samples were heated to 80° C. As the SNA:target complex was heated, the fluorescence increased due to dehybridization and subsequent dequenching of the FAM-tagged target. From the melting curves the melting temperature (Tm) the full width at half maximum FWHM of the transition, and maximum fluorescence intensity following melting were determined. The Tm was found to increase as n increased by up to about 12° C., suggesting that multiple segments were able to bind the target simultaneously. However, when n >4 a drop in Tm was observed. Note that one would not normally see a decrease in Tm as duplex lengthens for conventional linear DNA. Furthermore, the FWHM of the first derivative plots of the melting curves were determined. It was found that as n increased, the FWHM increased as well, indicating that the melting transition was less uniform. This suggests that as n increased there was a wider range of binding valencies present. By incubating with excess targets (25×), the number of targets bound per SNA was also determined. Generally, as n increased fewer targets were bound potentially due to each target occupying more segments on the particle surface.

Next, whether increased stringency, resulting in a lower Kd for each segment, would lead to higher valency binding was tested. The same series of melting experiments were repeated, with the exception of performing the hybridization in a more stringent buffer, 0.1×saline-sodium citrate buffer (SSC), 0.2% Tween 20 (Polyethylene glycol sorbitan monolaurate) (about 15 mM Na⁺). It was observed that the Tm's for n=1-6 SNAs increased by up to about 4° C. when hybridization stringency increased. The value of n giving the highest Tm also increased to n=6. Furthermore, in more stringent hybridization conditions, reversal of the trends for binding uniformity and capacity were seen, with n=4 resulting in the most uniform binding and maximum targets bound.

### HeteroMV SNAs Show Improved Binding Affinity to Target.

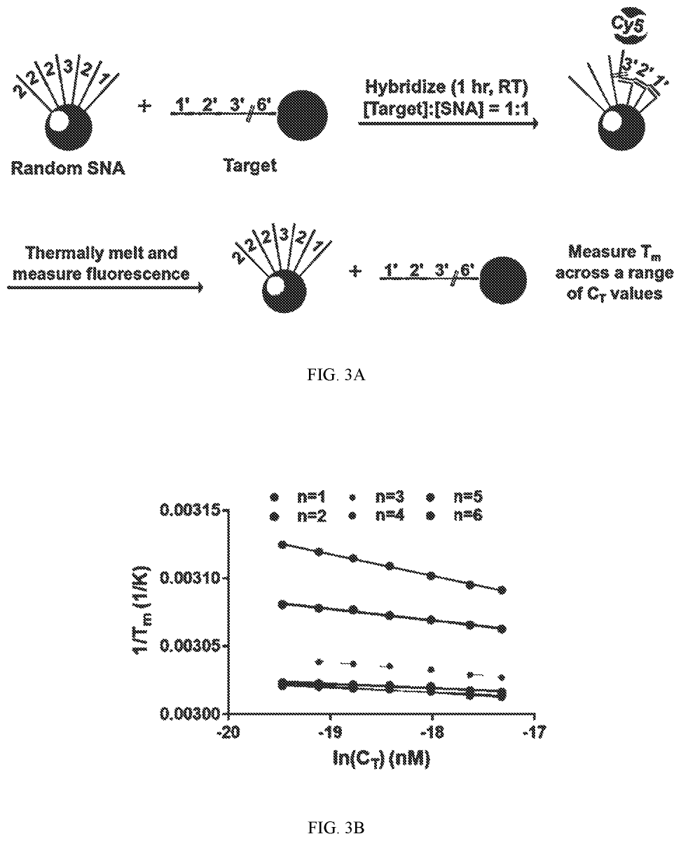

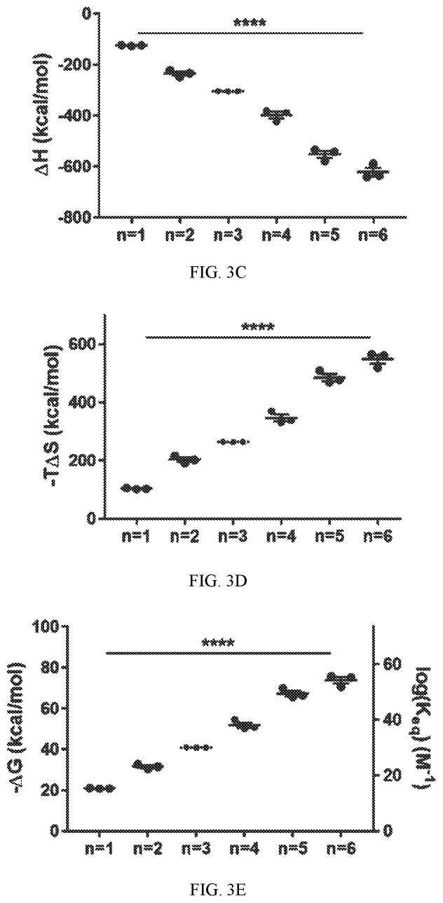

The full thermodynamic binding parameters of randomly organized n=1-6 heteroMV SNA binding to target were measured. In these studies, the particles were incubated at a 1:1 ratio of SNA to target for 1 hr (FIG. 3A). The concentration of SNA was varied from 1.75 to 15 nM and the Tm for the complex was measured as the cy5-labeled target was thermally dehybridized. For each SNA, about 20 thermal melting curves were used to populate the van′t Hoff plot showing In(Cr) versus 1/Tm (FIG. 3B). This was used calculate the ΔH and ΔS of binding, from which the ΔG of binding was then derived.

Note that Cr is the total concentration of SNA and target. Tm plateaued for SNAs with n >4. The flattening slopes as n increases correspond to a more favorable enthalpy of binding. On the other hand, the entropic cost of binding increased with increasing n. Nonetheless, the enthalpic benefits outweighed the entropic costs, resulting in an enhancement in ΔG with increasing n. Surprisingly, the improvement in affinity continued for the n=5 and 6 SNAs, potentially due to the 1:1 SNA:target ratio used for this experiment resulting in higher valency binding. The decrease in ΔG corresponds to an about 40 order-of-magnitude enhancement in binding affinity (Keq) and a multivalent enhancement value (B) of 7×10³⁸ as n increased from 1 to 6.

Development and Characterization of Patterned heteroMV SNAs.

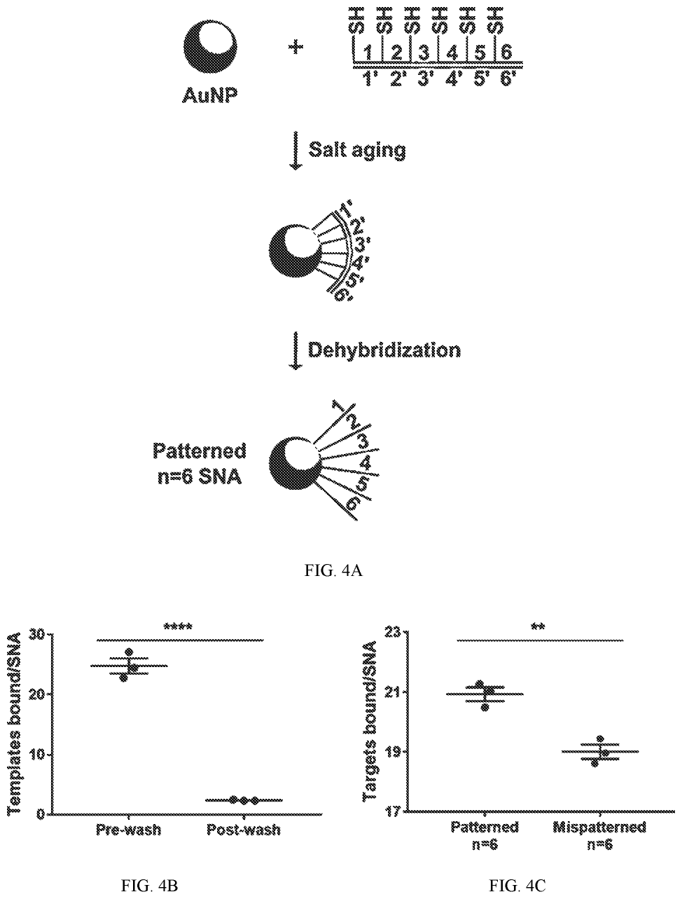

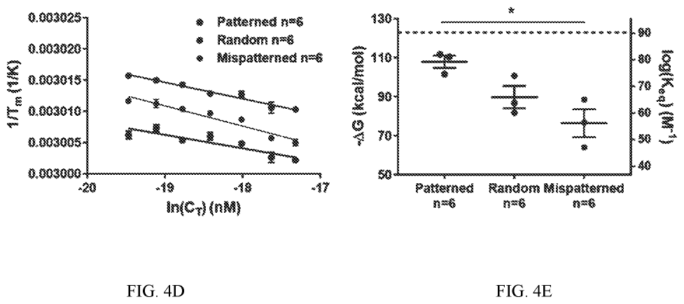

It is believed that spatial patterning of SNAs could boost target affinity compared to that of heteroMV SNAs with random oligo positioning. Experiments were performed to determine whether using a molecular printing method to create patterned heteroMV SNAs was beneficial (FIG. 4A). Segments 1-6 were first hybridized to a ssDNA template, identical to the target sequence except for omission of the 2 nt spacers, forming an 81mer duplex with 5 “nicks” located between each segment binding region. A native PAGE gel was performed to confirm the successful binding of the six segments to the template. The segment/template complex was then incubated with the AuNP, forming up to six thiol linkages in a linear array on the NP surface (as illustrated in FIG. 4A). The single strand nicks help accommodate the local curvature of the spherical nanoparticle surface. Next, salt-aging was performed to increase packing density of the complex on the particle. The template was then removed by dehybridization with a series of washes in DI water at RT. After template dehybridization, segments 1-6 remain on the particle surface because of the strong thiol-gold association, with controlled position and spacing, yielding a spatially patterned heteroMV SNA with n=6. The DNA-AuNP conjugate was characterized. Firstly, the number of complexes loaded to each particle was quantified by detecting release of a FAM-labeled template following heating. This melting assay revealed that ˜25 templates were bound to each SNA. Next, template dehybridization was validated. Approximately 90% of templates were removed. Moreover, the total number of binding ligands (segments 1-6) per particle was quantified to be about 135. This indicates that each template was bound to about 5.5 segments, offering further evidence of successful hybridization and loading onto the particle.

Impact of spatial patterning on heteroMV SNA binding.

To characterize target binding by patterned heteroMV SNAs, thermal melting curves were obtained. The van't Hoff relation was applied to determine thermodynamic binding constants. For these assays, mispatterned heteroMV SNAs were created to serve as an additional control. The mispatterned n=6 SNAs are chemically identical to patterned SNAs (same total number of segments 1-6) except for the relative positioning of the oligos on the NP surface, allowing the role of spatial patterning to be properly elucidated. First, the two particle types were incubated with excess of the non-spacer containing target (s=0 target) labeled with FAM, which is identical to the template used for patterning, and washed with high stringency buffer (0.1×SSC, 0.2% Tween20). Thermal melting curves were then obtained by measuring the fluorescence increase. While, the patterned and mispatterned SNAs demonstrated a similar melting temperature (<1° C. difference), patterned SNAs were able to bind a few more targets than their mispatterned counterparts (FIG. 4C). To more sensitively examine the effects of spatial patterning, the thermodynamics and affinity of the patterned binding interaction were quantified using the van′t Hoff relation and compared to the random SNAs. These experiments revealed a strong impact of oligo positioning on the binding enthalpy, as the dependence of Tm on concentration became less dramatic when going from intentionally mispatterned to random to patterned oligo positioning (FIG. 4D). This enthalpic enhancement with spatial patterning is likely the result of more segments binding each target and reduced strain on the binding entities. As before, the enthalpic benefits exceeded the entropic costs, leading to a significantly more favorable binding free energy and a ˜23 order-of-magnitude enhancement in Keq for patterned SNAs over mispatterned (FIG. 4E). Notably, the predicted ΔG for the same duplex containing no nicks is ˜−123 kcal/mol (dashed line in FIG. 4E), which is only ˜10% greater than the ΔG of the patterned SNA (˜108±3.2 kcal/mol) to the same target. These results demonstrate that proper positioning of the binding ligands on the NP surface is important for forming a highly effective multivalent interaction.

### Effect of Spacers in Target Sequence on Multivalent Binding.

The impact of spacers in the target were quantified by comparing the thermodynamics of binding in the absence of a spacer to that of binding in the presence of single- or di-nucleotide spacers between binding sites (s=0,1, or 2 target). First, the binding affinities of n=1-6 random SNA binding to the s=0 and s=1 targets were quantified through the van′t Hoff assay and compared to the s=2 data. As n increased, a trend of increasing binding affinity (more than 10 orders of magnitude when n=6) was observed as the spacer was removed from the target. This suggests that the oligo-oligo distance for random SNAs more closely matches the s-0 target, and additional target flexibility is counterproductive. Moreover, the impact of spacer length was compared for n=6 patterned, random, and mispatterned SNAs to determine if the same trend is true for particles with different ligand patterning. Note that the patterned particles are created using an s=0 template and therefore the oligo spacing should match the s-0 target. The affinity of patterned SNAs, with controlled ligand positioning, was significantly diminished as the spacer lengthened. These results demonstrate that the patterned SNAs offer a platform suited to interrogate the effects of mismatched ligand-receptor spacings on the binding avidity of multivalent interactions. Thus, the enthalpy and entropy of binding for patterned SNAs to s=0-2 targets were analyzed. Improper spacing led to diminished enthalpic benefits from multivalent binding as the ligands, in this case likely the T10 linker, are strained to optimally bind the target. Alternatively, increased spacer length did reduce the entropic cost of binding as more disorder was maintained following binding.

1. A method of attaching multiple single stranded block nucleic acids to a nanoparticle surface in close proximity in a sequential pattern comprising:
i) providing a nucleic acid complex comprising
1) a single stranded template nucleic acid having multiple template segments and
2) multiple single stranded block nucleic acids,
wherein the multiple single stranded block nucleic acids contain a first segment and a second segment providing multiple first block segments and multiple block second segments,
wherein the multiple first block segments are substantially variant sequences and hybridize with the multiple template segments, and
wherein the multiple second block segments comprise an anchor for attaching to the surface of the nanoparticle;
ii) mixing the nucleic acid complex with the nanoparticle under conditions such that the second block segments are conjugated to the nanoparticle surface providing a nanoparticle coated with the nucleic acid complex; and
iii) separating the target sequence from nucleic acid complex providing a nanoparticle coated with multiple single stranded block nucleic acids of substantially variant sequences in close proximity in a sequential pattern.

2. The method of claim 1 wherein separating the target sequence from nucleic acid complex is exposing the nanoparticle coated with the nucleic acid complex with increasing concentrations of buffered salt solutions providing a salt-aged nanoparticle coated with the nucleic acid complex; and exposing the salt-aged nanoparticle coated with the nucleic acid complex with a solution disrupting hybridization providing a nanoparticle coated with multiple single stranded block nucleic acids that are in close proximity in a sequential pattern.

3. The method of claim 1 wherein separating the target sequence from nucleic acid complex is exposing the nanoparticle coated with the nucleic acid complex to temperatures at or below 0 degrees Celsius providing a frozen nanoparticle coated with the nucleic acid complex; and exposing the frozen nanoparticle coated with the nucleic acid complex to a temperature above 0 degrees Celsius providing a frozen-thawed nanoparticle coated with the nucleic acid complex and exposing the frozen-thawed nanoparticle coated with the nucleic acid complex with a solution disrupting hybridization providing a nanoparticle coated with multiple single stranded block nucleic acids that are in close proximity in a sequential pattern.

4. The method of claim 1 wherein the multiple first block segments are three or more substantially variant sequences.

5. The method of claim 1 wherein the multiple single stranded block nucleic acids are less than 50 nucleotides in length.

6. The method of claim 1 wherein the multiple template segments are separated from each other by one, two, or more nucleobases on the contiguous single stranded template.

7. The method of claim 1 wherein the multiple template segments are separated from each other by less than four nucleobases on the contiguous single stranded template.

8. The method of claim 1 wherein the single stranded template nucleic acid comprises more than 50 nucleotides.

9. The method of claim 1 wherein the nanoparticle core has a diameter of less than 25, 50, or 100 nm.

10. The method of claim 1 wherein the anchor for attaching to the surface of the nanoparticle is thiol.

11. A method of detecting a single stranded nucleic acid comprising:
i) exposing a nanoparticle coated with multiple single stranded block nucleic acids made by the process of claim 1 to a sample suspected of comprising a single contiguous nucleic acid sequence of greater than 50 nucleotides having substantially the same sequence as the single stranded template nucleic acid and
ii) detecting hybridization of the single contiguous nucleic acid sequence to the nanoparticle coated with multiple single stranded block nucleic acids.

12. The method of claim 11, wherein having substantially the same sequence as the single stranded template nucleic acid is greater than 60%, 70%, 80%, 90%, 95%, 97%, 98% or more identity.

13. A nanoparticle surface comprising multiple single stranded block nucleic acids,
wherein the multiple single stranded block nucleic acids contain a first segment and a second segment providing multiple first block segments and multiple block second segments,
wherein the multiple first block segments contain substantially variant sequences and hybridize with multiple target segments, and
the multiple second block segments are attached to the surface of the nanoparticle.

14. The nanoparticle of claim 13 wherein the multiple target segments are contained with a single stranded nucleic acid.

15. The nanoparticle of claim 13 wherein the multiple single stranded block nucleic acids are in close proximity in a sequential pattern.

16. The nanoparticle of claim 13 wherein the multiple first bock segments are three or more substantially variant sequences.

17. The nanoparticle of claim 13 wherein the multiple single stranded block nucleic acids are less than 50 nucleotides in length.

18. The nanoparticle of claim 13 comprising a therapeutic agent.
